(19) United States
(12) Patent Application Publication
(10) Pub. No.: US 2013/0310420 A1
(43) Pub. Date: Nov. 21, 2013

(54) STABLE FORMULATIONS OF PITAVASTATIN

(71) Applicants: Yu-Chien Hsiao, San Diego, CA (US); Chi-Tai Chang, New Taipei City (TW); Che-Chin Lin, New Taipei City (TW)

(72) Inventors: Yu-Chien Hsiao, San Diego, CA (US); Chi-Tai Chang, New Taipei City (TW); Che-Chin Lin, New Taipei City (TW)

(73) Assignee: ORIENT PHARMA CO., LTD., Yunlin County (TW)

(21) Appl. No.: 13/865,118

(22) Filed: Apr. 17, 2013

Related U.S. Application Data
(60) Provisional application No. 61/635,237, filed on Apr. 18, 2012.

(51) Int. Cl. A61K 47/02; A61K 31/47

(52) U.S. Cl. CPC A61K 47/02; A61K 31/47
USPC 514/311

(57) ABSTRACT

Disclosed is a pharmaceutical composition comprising pitavastatin, or its salt or ester and MgO, of which the aqueous solution or dispersion has pH of from 10 to 10.8. The composition has good time-dependent stability and has no change in its outward appearance even after having been stored long.

### CROSS REFERENCE

This application claims the benefit of U.S. provisional application Ser. No. 61/635,237, filed Apr. 18, 2012, which is incorporated by reference in its entirety.

### BACKGROUND OF THE INVENTION

Pharmaceutical compositions which are unstable in an acidic or basic environment may require a basic or acidic excipient to enhance storage stability. However, the active ingredient in the compositions may comprise functional groups that are sensitive to both acidic and basic environments and neutral environments may be needed.

### SUMMARY OF THE INVENTION

In accordance with the present invention, the present invention provides basic pharmaceutical compositions comprising Pitavastatin that have excellent storage stability.

In one aspect, provided herein are pharmaceutical compositions consisting essentially of Pitavastatin, or its salt, solvate or ester, about 3% to about 20% of magnesium oxide by weight, and a pharmaceutically acceptable carrier.

In another aspect provides pharmaceutical compositions comprising Pitavastatin, or its salt, solvate or ester, about 3% to about 20% of magnesium oxide by weight, and a pharmaceutically acceptable carrier wherein said composition wherein an aqueous solution or dispersion of the pharmaceutical composition has pH of from 10.0 to 10.8.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

HMG-CoA reductase inhibitors having the formula R—CH═CH—CH(OH)—CH₂—CH(OH)—CH₂—COOH are thought to be unstable at low pH. For example, fluvastatin, which is not stable as a free acid, has been formulated with an alkaline medium, such as calcium carbonate or sodium carbonate, into preparations with pH of 8 or higher (U.S. Pat. No. 5,356,896). It is thought the extreme lability of the β-δ-hydroxy groups on the heptenoic acid chain and the presence of the double bond, such that at neutral to acidic pH, the compounds readily undergo decomposition reactions. However, it has been found that Pitavastatin (which contains the β-δ-hydroxy groups in the heptenoic acid chain) in the high pH formulations are still unstable and a stable formulation with pH 6.8 to 7.8 for Pitavastatin has been realized (see U.S. Pat. No. 6,465,477).

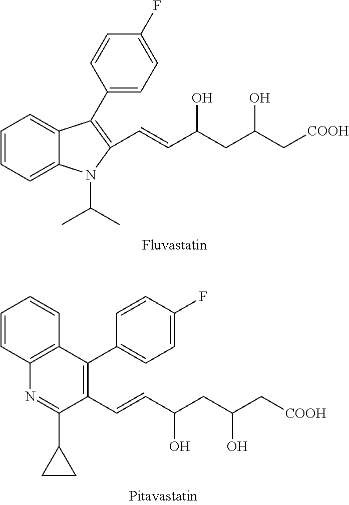 [SMILES: CC(C)n1c(/C=C/C(O)CC(O)CC(=O)O)c(-c2ccc(F)cc2)c2ccccc21 ; O=C(O)CC(O)CC(O)/C=C/c1c(C2CC2)nc2ccccc2c1-c1ccc(F)cc1]

In accordance with the present invention, it is found unexpectively a stable high pH formulation for Pitavastatin. In some embodiments provide pharmaceutical compositions consisting essentially of Pitavastatin, or its salt, solvate or ester, about 3% to about 20% of magnesium oxide by weight, and a pharmaceutically acceptable carrier. In certain embodiments, an aqueous solution or dispersion of said pharmaceutical composition has pH of from 10.0 to 10.8. In some embodiments, the composition does not include pH regulators such as such as L-arginine, sodium phosphate, disodium hydrogen phosphate, sodium dihydrogen phosphate, potassium phosphate, dipotassium hydrogen phosphate, potassium dihydrogen phosphate, disodium citrate, sodium succinate, ammonium chloride, and sodium benzoate.

In some embodiments, the composition further comprises at least one material selected from the group consisting of excipients, disintegrators, binders and lubricants. In certain embodiments, the excipients comprise microcrystalline cellulose, lactose, mannitol, starch, or combination thereof. In certain embodiments, the disintegrators comprise sodium starch glycolate, kollidon CL, croscarmellose sodium, hydroxypropyl cellulose, or combination thereof. In certain embodiments, the binders comprise Polyvinylpyrrolidone such as PVP K30.

In some embodiments, provided herein are pharmaceutical compositions comprising Pitavastatin, or its salt, solvate or ester, about 3% to about 20% of magnesium oxide by weight, and a pharmaceutically acceptable carrier wherein an aqueous solution or dispersion of said pharmaceutical composition has pH of from 10.0 to 10.8. In certain embodiments, the composition further comprises at least one material selected from the group consisting of excipients, disintegrators, binders and lubricants. In certain embodiments, the excipients comprise microcrystalline cellulose, mannitol, starch, lactose, or combination thereof. In certain embodiments, the disintegrators comprise sodium starch glycolate, kollidon CL, croscarmellose sodium, hydroxypropyl cellulose, or combination thereof. In certain embodiments, the binders comprise polyvinylpyrrolidone such as PVP K30.

The pharmaceutical composition of the present invention can be formulated into various forms of preparations. For example, the composition may be formulated into tablets, granules, powders, troches, capsules, chewables, film-coated preparations of these, and even sugar-coated preparations thereof.

Where the pharmaceutical composition of the present invention is formulated into such peroral solid preparations, any of excipients, binders, disintegrators and lubricants can be added thereto. An ordinary skilled in the art would readily recognize the desired or suitable excipients, binders, disintegrators and/or lubricants.

The excipients comprise, for example, lactose, starch (e.g., corn starch), denatured corn starch, mannitol, lactose, sorbitol, wood cellulose, microcrystalline cellulose, combination thereof, or the like. In certain embodiments, the excipients comprise microcrystalline cellulose, lactose, mannitol, starch, or combination thereof.

The binders comprise, for example, hydroxypropyl cellulose, hydroxypropylmethyl cellulose, polyvinylpyrrolidone, polyvinyl alcohol, and partial saponificates of these, which can be used either singly or as combined. In certain embodiments, the binders comprise polyvinylpyrrolidone such as PVP K30.

The disintegrators comprise, for example, low substituted hydroxypropyl cellulose, carmellose, sodium carboxy starch, calcium carmellose, sodium starch glycolate, kollidon CL, corn starch, partially-alphatized starch, Croscarmellose Sodium, Hydroxypropyl Cellulose, crospovidone (such as Crospovidone XL-10), combinations thereof, or the like. In certain embodiments, the disintegrators comprise sodium starch glycolate, kollidon CL, croscarmellose sodium, hydroxypropyl cellulose, or combination thereof.

The lubricants comprise, for example, magnesium stearate, stearic acid, palmitic acid, calcium stearate, talc, combination thereof, or the like.

The amounts of Pitavastatin constituting the composition of the present invention are not specifically defined. For example, the amount of Pitavastatin, its salt, solvate, or ester may be from 0.01 to 40% by weight, from 0.03 to 30% by weight, from 0.05 to 20% by weight, from 0.5 to 10% by weight, from 0.5 to 5% by weight; and magnesium oxide may be added to the composition in such an amount that is necessary for making the aqueous solution or dispersion of the composition have pH of from 10.0 to less than 11.0; from 10.0 to 10.8 or from 10.2 to 10.6. In some embodiments, the invention compositions comprise about 3% to about 30% of magnesium oxide by weight, about 3% to about 20% of magnesium oxide by weight, or about 5% to about 15% of magnesium oxide by weight.

In some embodiments the invention compositions are formulated for oral use. In some embodiments, the compositions comprise the excipient in an amount of from 30 to 95% by weight, the binder in an amount of from 1 to 20% by weight, the disintegrator in an amount of from 1 to 30% by weight, and the lubricant in an amount of from 0.5 to 10% by weight.

In some embodiments, any additional components, such as sweeteners, flavorings and colorants may also be added to the composition of the present invention.

In some embodiments,

### Certain Pharmaceutical and Medical Terminology

The term “acceptable” with respect to a formulation, composition or ingredient, as used herein, means having no persistent detrimental effect on the general health of the subject being treated.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical composition” refers to a mixture of a compound (i.e., Pitavastatin described herein) with other chemical components, such as, disintegrators, binders, lubricants, carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

In some embodiment, the pharmaceutical compositions described herein are formulated for oral administration. In various embodiments, the invention compositions described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipients with Pitavastatin described herein, optionally grinding the resulting mixture, and processing the mixture of granules, after adding suitable auxiliaries, if desired, to obtain tablets or dragee cores. An ordinary skill in the art may use suitable excipients, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents (disintegrators) are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of invention compositions described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In certain embodiments, invention pharmaceutical compositions are formulated in any conventional manner using one or more physiologically acceptable carriers comprising excipients and auxiliaries which facilitate processing of the active compound (i.e., Pitavastatin) into preparations which can be used pharmaceutically. Proper formulation is dependent upon the route of administration chosen. Any pharmaceutically acceptable techniques, carriers, and excipients is optionally used as suitable and as understood in the art. Invention pharmaceutical compositions comprising Pitavastatin may be manufactured in a conventional manner, such as, by way of example only, by means of conventional mixing, dissolving, granulating, dragee-making, levigating, emulsifying, encapsulating, entrapping or compression processes.

In certain embodiments, invention pharmaceutical composition may be formulated to aqueous suspensions including one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In certain embodiments, pharmaceutical aqueous suspension compositions are packaged in single-dose non-reclosable containers. Alternatively, multiple-dose reclosable containers are used, in which case it is typical to include a preservative in the composition.

All of the various embodiments or options described herein can be combined in any and all variations. The following Examples serve only to illustrate the invention and are not to be construed in any way to limit the invention.

### EXAMPLES

### Example 1

### Exemplary Pitavastatin Formulation

Pitavastatin, magnesium oxide and a fraction of the excipients (e.g., lactose) were mixed together for 2 to 10 minutes employing a suitable mixer. The resulting mixture was passed through a #12 to #40 mesh size screen. Microcrystalline cellulose, croscarmellose sodium and the remaining lactose were added and the mixture was mixed for 2 to 10 minutes. Thereafter, magnesium stearate (a lubricant) was added and mixing was continued for 1 to 3 minutes. The resulting homogeneous mixture was then compressed into tablets each containing 5 mg, 10 mg, 20 to 40 mg Pitavastatin. A dispersion of the tablets in water had a pH of about 10.

Exemplary formulations are shown below.

| | Formulation | | |
|---|---|---|---|
| | Sample 41 | Sample 42 | Sample 66 |
| Material | Percent by Weight | | |
| Pitavastatin | 1.6 | 1.6 | 1.6 |
| Mannitol | 49.7 | 46.5 | — |
| Lactose | — | — | 38.7 |
| Microcrystalline cellulose | 24.8 | 23.2 | 38.7 |
| MgO | 9.5 | 14.3 | 4.7 |
| Povidone | 2.9 | 2.9 | 2.9 |
| Sodium Starch Glycolate | — | — | 5.7 |
| Kollidon CL | — | — | 1.9 |
| Croscarmellose sodium | 4.8 | 4.8 | — |
| Magnesium stearate | 1.9 | 1.9 | 1.0 |
| Opadry II White | 4.8 | 4.8 | 4.8 |

### Example 2

### HPLC Conditions for Stability (Impurity) Test

Stability of invention formulation comprising Pitavastatin was analyzed by HPLC. The HPLC conditions are as follows:

| | |
|---|---|
| Buffer | Sodium 1-Heptanesulfonate (10 mM, adjust |
| | with H₃PO₄ to pH 3.0 |
| Mobile Phase | Buffer: Acetonitrile = 55:45 (v/v) |
| Column | Inertsil C8-3 (5 μm; 150 × 4.6 mm; GL Sciences Inc., |
| | Japan) with a guard column (Hichrom, HI-5C18) |
| Flow Rate | 1.0 mL/minutes |
| Wavelength | 248 nm |
| Column | 30° C. |
| Temperature | |
| Injection Volume | 50 μL |
| Run Time | 25 minutes |

Diluent. Add 20 mL of water to a 100 mL volumetric flask, and dilute with methanol to volume.

Standard Preparation. Weigh accurately about 25.0 mg of pitavastatin calcium working standard to a 25-mL amber volumetric flask. Add 5 mL of water, sonicate 1 minute. Add about 17 mL of methanol, sonicate 1 minute, and dilute with methanol to volume, and mix. Transfer 0.5 mL of this solution to a 25-mL amber volumetric flask, and dilute with Diluent to volume (20.0 ug/ml. of pitavastatin calcium), and mix.

System Suitability. Inject the Diluent to verify that there are no interfering peaks. Inject the Standard Preparation, and record the peak responses as directed for Procedure: the column efficiency is not less than 3000 theoretical plates; the tailing factor is not more than 2.0; and the relative standard deviation (RSD) for five replicate injections is not more than 2.0%.

Test Preparation. Randomized select 20 tablets, weigh and finely powder. Transfer an accurately weighed portion of the powder, equivalent to about 20 mg of pitavastatin calcium to a 100-mL amber volumetric flask. Add 20 mL of water, sonicate 5 minutes, and then add about 70 mL of methanol, sonicate 10 minutes, and then dilute with methanol to volume of flask, and mix. Transfer 5 mL of this solution to a 50-mL amber volumetric flask, and dilute with Diluent to volume of the flask (20.0 ug/ml. of pitavastatin calcium), and mix

Procedure. All the sample solutions first were filtered through the O.4S-llm polypropylene filter, discarding the first 1 mL of the filtrate, before HPLC analysis.

Separately inject equal volumes (50 ul.) of the Diluent, Standard Preparation and Test Preparation into the chromatograph, record the chromatograms for about 2.5 times the retention time of pitavastatin calcium, and measure the responses for all the peaks, disregarding any peaks corresponding to those obtained from the Diluent. Calculate the percentage of each impurity in the portion of Pitavastatin calcium sample taken by the formula:

Impurity (%)=(C_{S}/C_{U})×(R_{I}/Rs)×100%

in which C_{S} and C_{U} are the concentrations (in ug/ml.) of pitavastatin calcium in the Standard Preparation and the Test Preparation, respectively; R_{I} is the peak response of each individual impurity obtained from the Test Preparation; and Rs is the response from the major peak obtained from the Standard Preparation.

### Example 3

### Stability Test of Various Basic Formulations

Several basic tablets were prepared in accordance with the method described in Example 1 and were subject to a stability study at 55° C. or 40° C. under 75% relative humidity (75% RH). The results are summarized in the following tables.

To verify the prior art results, a few formulations in basic conditions (see Table 1, Sample-031, Sample-032, Sample-033 and Sample-038 were prepare in accordance with the prior art method and subject to the stability test at 55° C. at 75% RH. The stability test results are summarized in Table 2.

TABLE 1
Compositions of Samples 031, 032, 033, and 038

| Formulation | Composition | pH |
|---|---|---|
| Sample-031 | Calcium Carbonate- 25% | 9.00 |
| Sample-032 | TRIS (tromethamine)-1% | 8.62 |
| Sample-033 | TRIS (tromethamine)-4% | 9.46 |
| Sample-038 | Sodium Bicarbonate-10% | 8.38 |

TABLE 2
Stability test results of Samples 031, 032, 033,
and 038 at 55° C./75% RH

| | Impurity (55° C./75% RH) | | |
|---|---|---|---|
| Formulation | | Individual Maximum | Total |
| No: | Time | (NMT 0.2%) | (NMT 1.0%) |
| Sample-031 | 2 Weeks | 0.940% | 1.624% |
| Sample-032 | 2 Weeks | 0.518% | 0.867% |
| Sample-033 | 2 Weeks | 2.533% | 2.734% |
| Sample-038 | 2 Weeks | 0.509% | 0.772% |

The data clearly shows and verifies that formulations with these compositions (e.g., calcium or sodium carbonate, organic base such as TRIS (tromethamine)) at pH 8.38 to 9.0, are not stable.

The invention formulations comprising magnesium oxide (MgO) with high pH (Table 3, Samples 041, 042, and 066) were prepared and investigated along with Sample 044 which exhibits a similar pH profile (pH 10.46) having a magnesium base (i.e., magnesium hydroxide). Another magnesium base comprising formulation (e.g., Sample 043) with pH 7.04 was also prepared and tested as a reference.

TABLE 3
Compositions of Samples 041, 042, 043, 044, and 066

| Formulation | Composition | pH |
|---|---|---|
| Sample-041 | MgO 10% | 10.75 |
| Sample-042 | MgO 15% | 10.76 |
| Sample-043 | Magnesium Aluminum Oxide- | 7.04 |
| | 10% | |
| Sample-044 | Magnesium Hydroxide-10% | 10.46 |
| Sample-066 | MgO 5% | 10.35 |

It was unexpected found that, contrary to the previous findings; the invention formulations comprising MgO are stable even at high pH. See Table 4. For example, after two weeks at 55° C./75% RH, the impurity of individual maximum of Samples 041, 042 and 066 were all less than 0.06%. On the other hand, the impurity of individual maximum of Sample 044 (pH 10.46, Magnesium Hydroxide) was higher than 2.7% after two weeks under the same condition.

TABLE 4
Stability test results of Samples 041, 042, 043, 044,
and 066 at 55° C./75% RH

| | Impurity (55° C./75% RH) | | |
|---|---|---|---|
| | | Individual Maximum | Total |
| Formulation | Time | (NMT 0.2%) | (NMT 1.0%) |
| Sample-041 | 2 Weeks | 0.055% | 0.233% |
| Sample-042 | 2 Weeks | 0.052% | 0.217% |
| Sample-043 | 2 Weeks | 2.719% | 3.167% |
| Sample-044 | 2 Weeks | 0.215% | 0.401% |
| Sample-066 | 2 Weeks | 0.050% | 0.227% |

### Example 4

### Stability Test of Invention Formulations in Comparison with the Known Neutral Formulation

The exemplary formulations (e.g., Sample-041, Sample-042, and Sample-066) were further subject to a stability test in comparison with the known formulation, Livalo Tablet having pH of 6.8-7.8, at 55° C. under 75% relative humidity. The stability test results for up to 2 months are summarized in Table 5. Table 6 shows the corresponding HPLC analysis of the stability test results.

TABLE 5
Stability study of exemplary formulations
in comparison with Livalo

| | Impurity (55° C./75% RH) | | |
|---|---|---|---|
| Formulation | | Individual Maximum | Total |
| No: | Time | (NMT 0.2%) | (NMT 1.0%) |
| Livalo Tab(IR0O) | 2 Weeks | 0.329% | 0.624% |
| Sample -041 | 2 Months | 0.067% | 0.260% |
| Sample -042 | 2 Months | 0.063% | 0.263% |
| Sample -066 | 2 Weeks | 0.050% | 0.227% |

TABLE 6
HPLC analysis of the stability test results

| | | Individual impurity % | | | | |
|---|---|---|---|---|---|---|
| | | RT at | RT at | RT at | RT at | Total |
| | | about 3.2 | about 6.2 | about 8.0 | about 10.0 | Impurities |
| Sample | | min | min | min | min | % |
| Sample -041 | Initial | 0.049 | 0.019 | — | 0.035 | 0.156 |
| | 55° C./75% RH, 2 W | 0.055 | 0.047 | 0.012 | 0.031 | 0.233 |
| | 55° C./75% RH, 1 M | 0.059 | 0.037 | 0.009 | 0.025 | 0.200 |
| | 55° C./75% RH, 2 M | 0.067 | 0.038 | — | 0.031 | 0.260 |
| Sample -042 | Initial | 0.048 | 0.022 | — | 0.035 | 0.159 |
| | 55° C./75% RH, 2 W | 0.052 | 0.045 | 0.010 | 0.029 | 0.217 |
| | 55° C./75% RH, 1 M | 0.056 | 0.037 | 0.007 | 0.033 | 0.189 |
| | 55° C./75% RH, 2 M | 0.063 | 0.038 | — | 0.033 | 0.263 |
| Sample -066 | Initial | 0.054 | 0.043 | 0.012 | 0.093 | 0.279 |
| | 55° C./75% RH, 1 W | 0.056 | 0.079 | 0.021 | 0.062 | 0.305 |
| | 55° C./75% RH, 2 W | 0.050 | 0.044 | 0.018 | 0.039 | 0.227 |
| Reference | Initial | 0.000 | 0.027 | 0.062 | 0.049 | 0.149 |
| Formulation | 55° C./75% RH, 2 W | 0.020 | 0.329 | 0.153 | 0.074 | 0.624 |
| Livalo Tab | | | | | | |

### Example 5

### Stability Test Results of Invention Exemplary Formulations

To confirm the unexpected benefit of invention formulation comprising MgO, Samples F005, F006 and F007 were prepared in accordance of the procedure of Example 1 with the same composition (comprising 5% MgO, Lactose, Microcrystalline cellulose, Povidone, Sodium Starch Glycolate, Kollidon CL, Magnesium stearate, and Opadry II White, Table 7). The samples were further diluted with water to prepare the corresponding 5% of suspension of sample solutions and and were subject to stability study under the accelerate condition at 40° C., 75% relative humidity. The results are summarized in Table 8.

TABLE 7
pH and Compositions of Samples
P01B005, P01B006, and P01B007

| Formulation | Composition of MgO | pH |
|---|---|---|
| Sample- P01B005 | 5% MgO | 10.46 |
| Sample- P01B006 | 5% MgO | 10.54 |
| Sample- P01B007 | 5% MgO | 10.37 |

TABLE 8
Stability study of exemplary formulations at 40° C. under 75% relative humidity

| | | CONDITION 40° C./75% RH | | | | | |
|---|---|---|---|---|---|---|---|
| | | Batch No. | | | | | |
| | | P01B005 | | P01B006 | | P01B007 | |
| TEST INTERVAL | | Initial | 3 Months | Initial | 3 Months | Initial | 3 Months |
| Assay | 95.0- | 97.60% | 98.00% | 99.00% | 97.40% | 97.10% | 98.30% |
| | 105.0% of | | | | | | |
| | the label | | | | | | |
| | claim. | | | | | | |
| Purity | Individual: | 0.11% | 0.08% | 0.03% | 0.09% | 0.12% | 0.10% |
| | NMT 0.20% | | | | | | |
| | Total | 0.22% | 0.22% | 0.07% | 0.24% | 0.19% | 0.28% |
| | impurities: | | | | | | |
| | NMT 1.0%. | | | | | | |

The study results clearly shown and confirm that the invention formulations are stable at high pH environments.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A pharmaceutical composition consisting essentially of Pitavastatin, or its salt, solvate or ester, about 3% to about 20% of magnesium oxide by weight, and a pharmaceutically acceptable carrier.

2. The pharmaceutical composition of claim 1 wherein an aqueous solution or dispersion of the pharmaceutical composition has pH of from 10.0 to 10.8.

3. The pharmaceutical composition of claim 1, wherein said composition does not include pH regulators.

4. The pharmaceutical composition of claim 1 wherein the composition further comprises at least one material selected from the group consisting of excipients, disintegrators, binders and lubricants.

5. The pharmaceutical composition of claim 1 wherein the composition is formulated for oral application.

6. The pharmaceutical composition of claim 4 wherein the excipients comprise microcrystalline cellulose, lactose, mannitol, starch, or combination thereof.

7. The pharmaceutical composition of claim 4 wherein the disintegrators comprise sodium starch glycolate, kollidon CL, croscarmellose sodium, hydroxypropyl cellulose, or combination thereof.

8. The pharmaceutical composition of claim 4 wherein the binders comprise polyvinylpyrrolidone.

9. The pharmaceutical composition of claim 8 wherein the polyvinylpyrrolidone is PVP K30.

10. A pharmaceutical composition comprising Pitavastatin, or its salt, solvate or ester, about 3% to about 20% of magnesium oxide by weight, and a pharmaceutically acceptable carrier wherein an aqueous solution or dispersion of said pharmaceutical composition has pH of from 10.0 to 10.8.
